(19) United States
(12) Patent Application Publication
(10) Pub. No.: US 2024/0051824 A1
(43) Pub. Date: Feb. 15, 2024

(54) METHOD TO CONTROL SYNGAS COMPOSITION FROM AN ENGINE-BASED SYNGAS GENERATOR

(71) Applicant: Research Triangle Institute, Research Triangle Park, NC (US)

(72) Inventors: Sameer Paravathikar, Research Triangle Park, NC (US); John R. Carpenter, Research Triangle Park, NC (US)

(21) Appl. No.: 18/271,277

(22) PCT Filed: Jan. 7, 2022
(86) PCT No.: PCT/US2022/011635

§ 371 Date: Jul. 7, 2023

Related U.S. Application Data
(60) Provisional application No. 63/135,033, filed on Jan. 8, 2021.

(51) Int. Cl. C01B 3/36; C10G 2/00; C07C 29/151; C07C 41/09

(52) U.S. Cl. CPC C01B 3/36; C10G 2/30; C07C 29/1518; C07C 41/09; C01B 2203/062; C01B 2203/0283; C01B 2203/0475; C01B 2210/0051; C01B 2203/0405; C01B 2210/001; C01B 2203/0415; C01B 2210/0025; C01B 2210/0014; C01B 2203/042; C01B 2203/061; C01B 2203/1258

(57) ABSTRACT

The present disclosure provides a process for controlling syngas composition from an internal combustion engine-based syngas generator. While air is typically used as an oxidant, with nitrogen (N₂) as a diluent, this results in expensive downstream compression, and low feedstock conversion efficiencies. This disclosure provides CO₂ as a diluent to reduce N₂ concentration in the syngas. In some embodiments, the CO₂ diluent may be from either a biogas processing coupled with methanol, DME, and/or hydrocarbon production; or natural gas processing coupled with Fischer-Tropsch (FT) synthesis and/or other hydrocarbon synthesis.

### 1. CROSS REFERENCE TO RELATED APPLICATIONS

This application is a § 371 U.S. National Stage of International Application PCT/US2022/11635, filed Jan. 7, 2022, having Atty. Docket No. 121-93-PCT, which claims the benefit of U.S. Application No. 63/153,033, filed Jan. 8, 2021, Parvathikar et al., entitled “Method to Control Syngas Composition from an Engine-based Syngas Generator”, Atty. Dkt. No. 121-93-PROV, which is hereby incorporated by reference in its entirety.

### 2. FIELD

The present disclosure provides a process for controlling syngas composition from an internal combustion engine-based syngas generator. While air is typically used as an oxidant, with nitrogen (N₂) as a diluent, this results in expensive downstream compression, and low feedstock conversion efficiencies. This disclosure provides carbon dioxide (CO₂) as a diluent to reduce N₂ concentration in the syngas. In some embodiments, the CO₂ diluent may be from either a biogas processing coupled with methanol, dimethyl ether (DME), and/or hydrocarbon production; or natural gas processing coupled with methanol, DME, or Fischer-Tropsch (FT) synthesis, or other hydrocarbon production.

### 3. BACKGROUND

### 3.1. Introduction

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly nor impliedly admitted as prior art against the present disclosure.

Engine-based syngas generators use an internal combustion engine to partially oxidize feedstock such as, but not limited to methane (CH₄) or natural gas using air as an oxidant to produce syngas. The syngas, a mixture of carbon monoxide (CO) and hydrogen (H₂), is an intermediate that can then be used to produce a variety of chemicals, including methanol and Fischer-Tropsch (FT) liquids. The engine operates predominantly through partial oxidation requiring oxygen to convert the hydrocarbon feed to syngas.

Previous work has disclosed the inclusion of air, enriched air, or pure oxygen as the feed oxidant. For example, U.S. Pat. Nos. 9,909,491 and 9,919,776 (Bromberg et al.) disclose an engine to generate hydrogen-rich gas in a reformer for a liquid fuel manufacturing system. They suggest a gaseous hydrocarbon fuel, such as natural gas, and an oxidant, such as air, oxygen-enriched air or oxygen, as reactants. For operation with enriched air or oxygen as a reactant Bromberg et al. suggest using nitrogen or water as a diluent. The reference suggests “it is possible to use some of the cool syngas produced for dilution, or to use the tail gas from the process.” Alternatively, the engine may be run as a closed system with argon as a diluent.

U.S. Pat. No. 2,391,687 discloses an engine for producing syngas using 90-99% oxygen as a reactant.

PCT Publication No. WO2019/067341 (Carpenter et al.) discloses an internal combustion engine reactor for producing syngas and conditions for operating the reactor under fuel rich conditions.

Practically the operation of the engine to produce syngas requires the use of air or potentially a mildly enriched air (typically 35-38 mol. % oxygen) as an oxidant. The nitrogen (N₂) in the air or enriched air is necessary since it acts as a diluent to provide manageable in-cylinder temperature and pressure profiles as well as a stable flame upon ignition. The need for a diluent limits the use of other oxidants such as highly enriched air or pure or nearly pure oxygen (O₂). The resulting syngas typically consists of a large amount of nitrogen, ≥50%. Separation of the N₂ from the syngas is difficult and expensive. The typical solution is to carry the N₂ through any downstream syngas conversion and separate the products after the conversion. This results in a need to compress the N₂ as well as the syngas in order to achieve the desired operating conditions for any synthesis processes that follow the engine and more than doubles the capital and operating costs of the system. Carrying inert N₂ through these stages results in high compression costs, large reactor volumes, and lower efficiencies in separations resulting in low carbon conversion.

### 4. SUMMARY OF THE DISCLOSURE

The present disclosure provides a method for producing syngas which comprises reacting a hydrocarbon fuel and enriched-oxygen containing feed gas in internal combustion engine reactor wherein the feed gas comprises a carbon dioxide diluent present at about 5 to about 50 mol. % and the enriched-oxygen is present about 25 to 95% mol. % so as to produce the syngas.

The disclosure also provides a system for the conversion of biogas to liquids, demonstrated here using methanol and/or DME as an example, a system which comprises (a) a biogas processing unit removing a substantial portion of sulfur compounds from the biogas and, optionally removing at least a portion of carbon dioxide from the biogas, to generate a clean biogas stream with about 1 to about 35 mol. % carbon dioxide content; (b) an air separation unit to generate an oxygen-rich gas stream with about 25 to about 95 mol. % oxygen; (c) an internal combustion engine reactor fluidly connected to the biogas processing unit and the air separation unit so as to react the clean biogas stream with the oxygen enriched stream so as to produce a syngas stream; (d) a gas separation unit and a syngas compression unit fluidly connected to the syngas stream from the internal combustion engine reactor so as to generate a processed syngas stream; and (e) a methanol, DME, and/or hydrocarbon synthesis unit fluidly connected to the processed syngas stream.

In addition, the disclosure provides a system for the conversion of natural gas to liquids, demonstrated here using synthetic crude oil as an example, a system which comprises (a) a natural gas fluid stream; (b) an air separation unit to generate an oxygen-rich gas stream with about 25 to about 95 mol. % oxygen; (c) an internal combustion engine reactor fluidly connected to the natural gas source and the air separation unit so as to react the clean biogas stream with the oxygen enriched stream so as to produce a syngas stream; (d) a water gas shift/gas separation unit and a syngas compression unit fluidly connected to the syngas stream from the internal combustion engine reactor so as to generate a processed syngas stream and a carbon dioxide rich stream; (e) wherein the carbon dioxide rich stream is fluidly connected to the internal combustion engine reactor to provide a carbon dioxide containing diluent stream; and (f) a Fischer Tropsch (FT) reactor fluidly connected to the processed syngas stream.

### 5. BRIEF DESCRIPTION OF THE FIGURES

FIG. 1 shows a block flow diagram for a biogas-based feedstock to provide the CO₂ diluent to produce methanol, DME, and/or hydrocarbons.

FIG. 2 shows a block flow diagram for FT synthesis from natural gas.

### 6. DETAILED DESCRIPTION OF THE DISCLOSURE

This disclosure provides methods and systems for providing CO₂ as a diluent in the feed to the engine. As discussed above, U.S. Pat. Nos. 9,909,491 and 9,919,776 disclose adding small amounts of other components such as steam, argon, or hydrogen to enable engine operation and using air or enriched air as the oxidant. Previously, air separation costs were prohibitive thereby limiting the use of pure O₂ or dilution of the O₂ with an alternative gas. Similarly, emerging feedstocks such as biogas already have significant CO₂ concentrations. CO₂ has the advantage of being easy to separate from syngas and not participating in partial oxidation reactions. However, at high temperatures, such as those seen in partial oxidation reaction, the CO₂ can participate in reforming reactions and water-gas shift reactions which will change the syngas composition. The short residence time and fast cooling to lower temperatures from peak in-cylinder temperatures limit these reactions of CO₂. By replacing the N₂ dilution with CO₂ dilution, a more valuable and higher quality syngas can be generated. The higher quality syngas enables cheaper and more efficient downstream conversions of the syngas to other desired products. The disclosure described herein includes:
Use of an O₂ stream (25 to 95 mol. % and up) to feed the syngas generator, this O₂ stream can come from any number of known sources, e.g. vacuum pressure swing adsorption, pressure swing adsorption, cryogenic separation, pipeline supplies, bulk liquid supply, etc. Quantities are referenced throughout this disclosure as mole fraction (percentage by moles, mol. %), one skilled in the art will recognize this term may be used interchangeably with percentage by volume or vol. %. Similarly, one may use the molecular weight of a gas of interest to convert, mol. % or vol. % to weight percent (wt. %).
Feed of a gaseous hydrocarbon stream which can be from numerous sources such as gas wells, associated gas from oil wells, waste gas streams, biogas from anaerobic digestion, biogas from landfills, etc.
Feed of a CO₂ stream from multiple sources to the engine system to dilute the total gas feed to the engine piston, including.
CO₂ already mixed with the fuel such as in the case of biogas or other potential waste streams.
CO₂ separated from the produced syngas prior to conversion that can be recycled to the engine feed.
CO₂ produced in the syngas conversion that can be recycled to the engine feed.
Any alternative source of CO₂ could be used to dilute the engine feed.
Start-up of the syngas generator can be envisioned under two circumstances—when CO₂ for dilution is available or is not available.
When CO₂ is not available, the syngas generator will be started under conditions similar to power generation where fuel-air mixture is near stoichiometric to lean producing more complete combustion with a CO₂-rich exhaust. CO₂ from this exhaust will be separated and recycled to begin feed dilution and shifting operation parameters from start-up conditions to syngas production conditions.
When CO₂ for dilution is available such as in the biogas fuel feed or from alternative sources, syngas generation system start-ups will begin with CO₂ dilution and proceed with the operational parameters to shift from combustion to partial oxidation during start-up.
Downstream of the engine, the produced syngas will then be conditioned to adjust the syngas composition as desired, such as (a) incorporation of a water-gas shift reactor to convert excess CO₂ to CO, adjust temperature and pressure as desired and/or (b) to separate excess CO₂ from the syngas to produce a higher concentration of CO and H₂ in the syngas than previous systems utilizing internal combustion engines in syngas generation systems.
Addition of unutilized H₂ from syngas conversion processes or other sources are envisioned as methods for adjusting the feed properties to balance some of the potential negative consequences such as lower flame speed that increasing CO₂ concentration in the feed can cause.

In some embodiments, the CO₂ diluent may be from either a biogas processing coupled with methanol, dimethyl ether (DME), or hydrocarbon production; or natural gas processing coupled with methanol, DME, or Fischer-Tropsch (FT) synthesis, or other hydrocarbon production. The hydrocarbons may be lower olefins (C2-C4 olefins), liquid fuels (C5-C20 hydrocarbons), or aromatics. Non-limiting examples include the following: For liquid fuels, see N. Duyckaerts, M. Bartsch, I. T. Trotu, N. Pfander, A. Lorke, F. Schuth, G. Prieto, Angew. Chem. Int. Ed. 2017, 56, 11480-11484 (Co/Al₂O₃ FT catalyst with Pt/ZSM-5 hydrotreating catalyst to form liquid hydrocarbons); J. Kang, K. Cheng, L. Zhang, Q. Zhang, J. Ding, W. Hua, Y. Lou, Q. Zhai, Y. Wang, Angew. Chem. Int. Ed. 2011, 50, 5200-5203 (mesoporous zeolite supported ruthenium nanoparticles to prepare C5-C11 isoparafins); X. Peng, K. Cheng, J. Kang, B. Gu, X. Yu, Q. Zhang, Y. Wang, Angew. Chem. Int. Ed. 2015, 54, 4553-4556 (zeolite supported cobalt nanoparticles to make C10-C20 hydrocarbons (diesel fuel). For lower olefins, see Cheng, B. Gu, X. Liu, J. Kang, Q. Zhang, Y. Wang, Angew. Chem. Int. Ed. 2016, 55, 4725-4728 Zr—Zn binary oxide and zeolite SAPO-34 to form CH₃OH, DME, C2-C4 olefins); F. Jiao, X. Liu, K. Gong, Y. Chen, G. Li, X. Bao, Angew. Chem. Int. Ed. 2018, 57, 4692-4696 (ZnCrOₓ-mordenite to form ethylene); X. Liu, W. Zhou, Y. Yang, K. Cheng, J. Kang, L. Zhang, G. Zhang, X. Min, Q. Zhang, Y. Wang, Chem. Sci. 2018, 9, 4708-4718 (Zn-doped ZrO2 nanoparticles and zeolite SSZ-13 nanocrystals to form C2-C4 olefins); J. Su, D. Wang, Y. Wang, H. Zhou, C. Liu, S. Liu, C. Wang, W. Yang, Z. Xie, M. He, ChemCatChem 2018, 10, 1536-1541 (Zirconium-doped Indium catalysts and SAPO-34 zeolite to form light olefins). For aromatics, see K. Cheng, W. Zhou, J. Kang, S. He, S. Shi, Q. Zhang, Y. Pan, W. Wen, Y. Wang, Chem 2017, 3, 334-347 and summary in Brosius, R., Claeys, M. Chem 2017, 3, 198-210 (Zn—ZrO₂ nanoparticles in zeolite to form aromatics); J. L. Weber, I. Dugulan, P. E. de Jongh, K. P. de Jong, ChemCatChem 2018, 10, 1107-1112 (iron-based FT catalysts and zeolites to synthesize aromatics and olefins); P. Zhang, L. Tan, G. Yang, N. Tsubaki, Chem. Sci. 2017, 8, 7941-7946 (Cr/Zn hybrid zeolite catalysts to form xylenes including para-xylene).

### 6.1. Definitions

While the following terms are believed to be well understood by one of ordinary skill in the art, the following definitions are set forth to facilitate explanation of the presently disclosed subject matter.

The term “partial oxidation” is understood to mean reacting a hydrocarbon with an oxidant at a level lower than the stoichiometric amount required for complete conversion to carbon dioxide and water.

Throughout the present specification, the terms “about” and/or “approximately” may be used in conjunction with numerical values and/or ranges. The term “about” is understood to mean those values near to a recited value. For example, “about 40 [units]” may mean within ±25% of 40 (e.g., from 30 to 50), within ±20%, ±15%, ±10%, ±9%, ±8%, ±7%, ±6%, ±5%, ±4%, ±3%, ±2%, ±1%, less than ±1%, or any other value or range of values therein or there below. Alternatively, depending on the context, the term “about” may mean±one half a standard deviation, ±one standard deviation, or ±two standard deviations. Furthermore, the phrases “less than about [a value]” or “greater than about [a value]” should be understood in view of the definition of the term “about” provided herein. The terms “about” and “approximately” may be used interchangeably.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

As used herein, the verb “comprise” as used in this description and in the claims and its conjugations are used in its non-limiting sense to mean that items following the word are included, but items not specifically mentioned are not excluded.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. The present disclosure may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

Unless defined otherwise, all technical and scientific terms used herein have the same meanings as commonly understood by one of ordinary skill in the art to which this disclosure belongs. Preferred methods, devices, and materials are described, although any methods and materials similar or equivalent to those described herein can be used in the practice or testing of the present disclosure. All references cited herein are incorporated by reference in their entirety.

The following Examples further illustrate the disclosure and are not intended to limit the scope. In particular, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

### 7. EXAMPLES

### 7.1. Example 1: Biogas Feedstock

Biogas typically consists of ˜65% CH₄, ˜35% CO₂ along with some sulfur compounds. Conventional usage of biogas involves removal of the sulfur compounds and CO₂, which results in a near-pipeline quality CH₄ stream (CO₂ less than 2 mol. %) which then can be used in the engine using air as an oxidant. In this scenario utilizing the present disclosure, the CO₂ in the biogas is used as a diluent in place of N₂ in the feed to the engine. This enables the use of enriched air with O₂ concentration from 25-95 mol %, provided by commercially available technologies including membranes and vacuum pressure swing adsorption (VPSA). CO₂ being relatively inert in CH₄ partial oxidation, and easier to separate from gas streams than N₂, allows for adjustment of syngas composition from the engine effluent to desired concentrations by removing CO₂ using one of several methods such as a membrane, prior to compression for further synthesis. In the instance of methanol synthesis, for example, the CO₂ is adjusted down to ˜8-12 mol %.

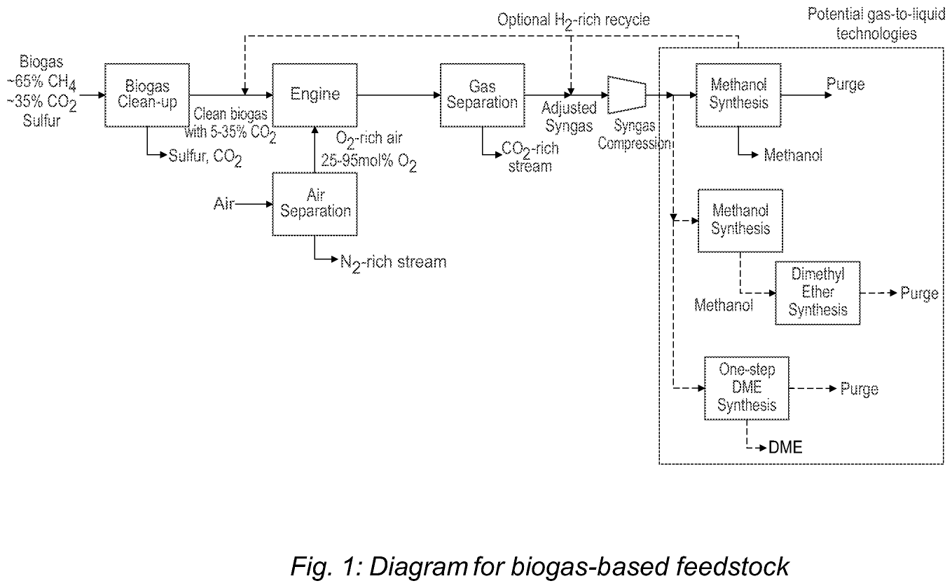

Another example of this scenario is the optional recycle of an H₂-rich stream, separated from the reactor tail gas using a selective membrane or a pressure swing adsorber (PSA), into the engine feed. This has the advantage of increasing the calorific value of the feed gas, which allows for stable flame propagation under conditions of high feed CO₂ concentrations. A block flow diagram of the proposed process is shown in FIG. 1.

### 7.2. Example 2: Natural Gas Feed

In the case of natural gas feed, such as those originating from pipelines, or stranded resources, the feedstock typically does not consist of high amounts of CO₂ or other suitable inerts as in the biogas scenario. Integration with a chemicals production process such as FT liquids, however, enables an opportunity to substitute the N₂ in the oxidant air with a different stream provided by downstream gas separation.

In the FT process, syngas is converted to synthetic crude in a catalytic reactor. The liquid products from the reaction, consisting of C5+ hydrocarbons, are separated from the gaseous products and unreacted feed, which include H₂, CO, CO₂, and light hydrocarbons.

In conventional FT process trains, CO₂ is removed in a solvent wash process, and an H₂-rich stream is subsequently recovered from the CO₂-free stream to be used in upgrading the C5+ crude stream.

This scenario exemplifies how a gas separation process downstream of the syngas conversion (FT synthesis reactor in this case) can be used to provide the CO₂ for dilution and improved overall system efficiency. In this scenario an H₂-rich stream is extracted from the gaseous effluent of the FT synthesis reactor, in a separation process such as a membrane or PSA. This generates a waste gas stream that is rich in CO₂ and hydrocarbons (labeled CO₂-laden stream) as well as at least one H₂-rich stream.

The waste gas stream rich in CO₂ and light hydrocarbons (labeled CO₂-laden stream), is recycled back to the engine feed, where the CO₂ acts as the diluent, and the other hydrocarbons can undergo partial oxidation, while helping stabilize in-cylinder ignition and combustion. Recycling this portion of the FT synthesis effluent back to the engine might require gas cooling, water-knock-off, additional filtration and small purge as are known in typical recycle setups. Recycling of the waste hydrocarbons also leads to greater overall system efficiency as those molecules have additional opportunity to be converted to desired product.

The H₂-rich streams can be used in multiple ways such as for further upgrading of crude to diesel/naphtha as shown. The H₂-rich stream can also be recycled to the syngas between the engine and FT synthesis to increase the H₂/CO ratio of the syngas feeding the FT block. Also, if necessary, and depending on the process train implemented, a small fraction of the H₂-rich stream can be recycled back to the engine feed for flame stabilization.

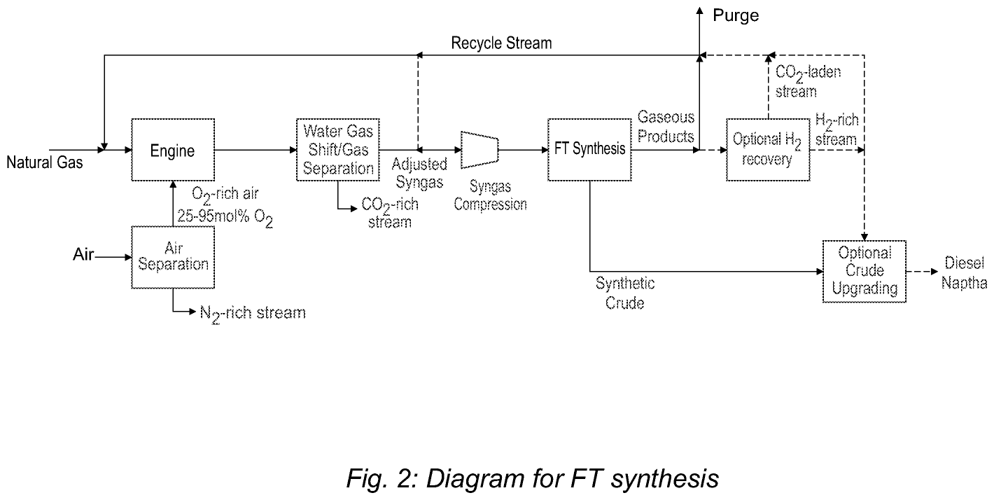

As described in Example 1, the composition of the syngas may be adjusted either by gas conversion through water-gas shift, separation, or a combination of the two, prior to compression for synthesis. An example incorporating this aspect of the disclosure is shown in FIG. 2. The use of this recycle allows for the replacement of N₂ as a diluent with the use of enriched air, in the feed to the engine. The extent to which the use of N₂ for dilution can be eliminated is dependent on catalyst performance and operating conditions of the FT synthesis, which affects CO₂ selectivity in the FT reaction, which could affect availability of the alternate inert gas streams.

### 8. REFERENCES

Bromberg, L., Green, W. H., Sappok, A., Cohn, D. R., Jalan, A., “Engine reformer systems for lower cost, smaller scale manufacturing of liquid fuels”, U.S. Pat. No. 9,909,491B2
Browne, J. B., “A techno-economic and environmental analysis of a novel technology utilizing an internal combustion engine as a compact, inexpensive micro-reformer for a distributed gas-to-liquids system”, Doctoral Thesis, Columbia University, 2016.

### 9. GENERALIZED STATEMENTS OF THE DISCLOSURE

The following numbered statements provide a general description of the disclosure and are not intended to limit the appended claims.

Statement 1: A method for producing syngas which comprises reacting a hydrocarbon fuel and enriched-oxygen containing feed gas in internal combustion engine reactor wherein the feed gas comprises a carbon dioxide diluent present at about 5 to about 50 mol. % and the enriched-oxygen is present about 25 to about 95% mol. % so as to produce the syngas. The carbon dioxide diluent may be present in about 5 to about 10 mol. %, about 10 to about 15 mol. %, about 15 to about 20 mol. %, about 20 to about 25 mol. %, about 25 to about 30 mol. %, about 30 to about 35 mol. %, about 35 to about 40 mol. %, about 40 to about 45 mol. %, or about 45 to about 50 mol. %. The enriched oxygen may be present in about 25 to about 35% mol. %, about 35 to about 45% mol. %, about 45 to about 55% mol. %, about 55 to about 65% mol. %, about 65 to about 75% mol. %, about 75 to about 85% mol. %, or about 85 to about 95% mol. %.

Statement 2: The method of statement 1, wherein the enriched-oxygen feed gas is obtained by vacuum pressure swing adsorption, pressure swing adsorption, cryogenic separation, permeable membrane gas separation, or a combination thereof.

Statement 3: The method of any of statements 1 or 2, wherein the hydrocarbon fuel is a gaseous hydrocarbon fuel.

Statement 4: The method of statement 3, wherein the gaseous hydrocarbon fuel is natural gas.

Statement 5: The method of statement 3, wherein the gaseous hydrocarbon fuel is a biogas.

Statement 6: The method of statement 3, wherein the gaseous hydrocarbon fuel is from a gas well, or an associated gas from an oil well.

Statement 7: The method of statement 3, wherein the gaseous hydrocarbon fuel is a fuel mixture comprising at least a portion of the carbon dioxide diluent.

Statement 8: The method of statement 7, wherein the fuel mixture is a biogas from a landfill or a biogas from anaerobic digestion.

Statement 9: The method of any of statements 1-8, wherein at least a portion of the carbon dioxide diluent is obtained by separation from the syngas downstream from the internal combustion engine reactor prior to a syngas processing step.

Statement 10: The method of any of statements 1-8, where the carbon dioxide diluent is obtained from an output of a syngas processing step.

Statement 11: The method of statement 1, where the carbon dioxide diluent is obtained from a separate source.

Statement 12: The method of any of statements 1-11, wherein the feed gas further comprises hydrogen and hydrocarbons added to increase the flame speed.

Statement 13: The method of statement 12, wherein the hydrogen and hydrocarbons added are obtained from a syngas conversion system.

Statement 14: The method of any of statements 1-13, wherein the internal combustion engine reactor is run under initially under a stoichiometric to lean fuel-oxygen ratio and then shifted to a rich fuel-oxygen ratio so as to maximize the production of syngas.

Statement 15: The method of any of statements 1-13, wherein carbon dioxide from the internal combustion engine reactor is initially run in a full oxidation mode so as to produce carbon dioxide, the carbon dioxide is separated, and is added to the feed gas stream.

Statement 16: The method of any of statements 1-15, wherein the syngas is conditioned by a water-gas shift reactor to convert excess carbon dioxide to carbon monoxide, to adjust the temperature, to adjust the pressure, to separate the excess carbon dioxide, or a combination thereof.

Statement 17: The method of any of statements 1-15, wherein the syngas is conditioned by separating the excess carbon dioxide or diluent by a membrane, a pressure swing adsorber, a solvent-based separation system, or a combination thereof.

Statement 18: The method of any of statements 1-17, wherein the syngas is converted to methanol in a methanol synthesis unit.

Statement 19: The method of claim 18, wherein the methanol is subsequently converted to dimethyl ether (DME) in a two-step DME synthesis unit.

Statement 20: The method of any of statements 1-17, wherein the syngas is directly converted to dimethyl ether (DME) in a one-step DME synthesis unit.

Statement 21: The method of any of statements 1-17, wherein the syngas is converted to a lower olefin, a liquid fuel, or an aromatic in a hydrocarbon synthesis unit.

Statement 22: The method of any of statements 1-17, wherein the syngas is converted to synthetic crude oil in a Fischer Tropsch (FT) reactor.

Statement 23: A system for the conversion of biogas to methanol which comprises (a) a biogas processing unit removing a substantial portion of sulfur compounds from the biogas and, optionally removing at least a portion of carbon dioxide from the biogas, to generate a clean biogas stream with about 1 to about 35 mol. % carbon dioxide content; (b) an air separation unit to generate an oxygen-rich gas stream with about 25 to about 95 mol. % oxygen content; (c) an internal combustion engine reactor fluidly connected to the biogas processing unit and the air separation unit so as to react the clean biogas stream with the oxygen enriched stream so as to produce a syngas stream; (d) a gas separation unit and a syngas compression unit fluidly connected to the syngas stream from the internal combustion engine reactor so as to generate a processed syngas stream; and (e) a methanol, DME, and/or hydrocarbon synthesis unit fluidly connected to the processed syngas stream.

Statement 24: The system of statement 23, wherein the biogas separation unit produces a clean biogas with about 5 to about 30 mol. % carbon dioxide content. The clean biogas may be about 5 to about 10 mol. %, about 10 to about 15 mol. %, about 15 to about 20 mol. %, about 20 to about 25 mol. %, about 25 to about 30 mol. %, about 30 to about 35 mol. % carbon dioxide content. The air separation unit may produce enriched oxygen with about 25 to about 35% mol. %, about 35 to about 45% mol. %, about 45 to about 55% mol. %, about 55 to about 65% mol. %, about 65 to about 75% mol. %, about 75 to about 85% mol. %, or about 85 to about 95% mol. % oxygen content.

Statement 25: The system of any of statements 23-24, wherein the gas separation unit is fluidly connected to the internal combustion engine reactor to introduce carbon dioxide as a diluent in the internal combustion engine reactor.

Statement 26: The system of any of statements 23-25, where the methanol, DME, and/or hydrocarbon synthesis unit produces a hydrogen stream and the hydrogen stream is fluidly connected to the internal combustion engine reactor, the gas separation unit, the syngas compression unit, or a combination thereof.

Statement 27: A system for the conversion of natural gas to synthetic crude oil which comprises (a) a natural gas fluid stream; (b) an air separation unit to generate an oxygen-rich gas stream with about 25 to about 95 mol. % oxygen; (c) an internal combustion engine reactor fluidly connected to the natural gas source and the air separation unit so as to react the clean biogas stream with the oxygen enriched stream so as to produce a syngas stream; (d) a water gas shift/gas separation unit and a syngas compression unit fluidly connected to the syngas stream from the internal combustion engine reactor so as to generate a processed syngas stream and a carbon dioxide rich stream; (e) wherein the carbon dioxide rich stream is fluidly connected to the internal combustion engine reactor to provide a carbon dioxide containing diluent stream; and (f) a Fischer Tropsch (FT) reactor fluidly connected to the processed syngas stream. The air separation unit may produce enriched oxygen with about 25 to about 35% mol. %, about 35 to about 45% mol. %, about 45 to about 55% mol. %, about 55 to about 65% mol. %, about 65 to about 75% mol. %, about 75 to about 85% mol. %, or about 85 to about 95% mol. % oxygen content.

Statement 28: The system of statement 27, wherein the water gas shift/gas separation unit is fluidly connected to the internal combustion engine reactor to introduce carbon dioxide as a diluent for the internal combustion engine reactor.

Statement 29: The system of any of statements 27-28, where the Fischer Tropsch (FT) reactor, the water gas shift/gas separation unit, the syngas compression unit, or a combination thereof, produces a hydrogen stream and the hydrogen stream is fluidly connected to a feed gas stream for the internal combustion engine reactor.

Statement 30: The system of any of statements 27-28, wherein the synthetic crude from the Fischer Tropsch (FT) reactor is fluidly connected to a crude upgrading unit and the crude upgrading unit produces diesel and/or naphtha.

It should be understood that the above description is only representative of illustrative embodiments and examples. For the convenience of the reader, the above description has focused on a limited number of representative examples of all possible embodiments, examples that teach the principles of the disclosure. The description has not attempted to exhaustively enumerate all possible variations or even combinations of those variations described. That alternate embodiments may not have been presented for a specific portion of the disclosure, or that further undescribed alternate embodiments may be available for a portion, is not to be considered a disclaimer of those alternate embodiments. One of ordinary skill will appreciate that many of those undescribed embodiments, involve differences in technology and materials rather than differences in the application of the principles of the disclosure. Accordingly, the disclosure is not intended to be limited to less than the scope set forth in the following claims and equivalents.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world. It is to be understood that, while the disclosure has been described in conjunction with the detailed description, thereof, the foregoing description is intended to illustrate and not limit the scope. Other aspects, advantages, and modifications are within the scope of the claims set forth below. All publications, patents, and patent applications cited in this specification are herein incorporated by reference as if each individual publication or patent application were specifically and individually indicated to be incorporated by reference.

What is claimed is:

1. A method for producing syngas which comprises reacting a hydrocarbon fuel and enriched-oxygen containing feed gas in internal combustion engine reactor wherein the feed gas comprises a carbon dioxide diluent present at about 5 to about 50 mol. % and the enriched-oxygen is present about 25 to 95% mol. % so as to produce the syngas.

2. The method of claim 1, wherein the enriched-oxygen feed gas is obtained by vacuum pressure swing adsorption, pressure swing adsorption, cryogenic separation, permeable membrane gas separation, or a combination thereof.

3. The method of claim 1, wherein the hydrocarbon fuel is a gaseous hydrocarbon fuel.

4. The method of claim 3, wherein the gaseous hydrocarbon fuel is natural gas, a biogas, or from a gas well, or an associated gas from an oil well.

5. (canceled)

6. (canceled)

7. The method of claim 3, wherein the gaseous hydrocarbon fuel is a fuel mixture comprising at least a portion of the carbon dioxide diluent.

8. The method of claim 7, wherein the fuel mixture is a biogas from a landfill or a biogas from anaerobic digestion.

9. (canceled)

10. The method of claim 1, where the carbon dioxide diluent is obtained from an output of a syngas processing step.

11. (canceled)

12. The method of claim 1, wherein the feed gas further comprises hydrogen and hydrocarbons added to increase the flame speed.

13. The method of claim 12, wherein the hydrogen and hydrocarbons added is obtained from a syngas conversion system.

14. The method of claim 1, wherein the internal combustion engine reactor is run under initially under a stoichiometric to lean fuel-oxygen ratio and then shifted to a rich fuel-oxygen ratio so as to maximize the production of syngas.

15. The method of claim 14, wherein carbon dioxide from the internal combustion engine reactor is initially run in a full oxidation mode so as to produce carbon dioxide, the carbon dioxide is separated, and is added to the feed gas stream.

16. The method of claim 1, wherein the syngas is conditioned by a water-gas shift reactor to convert excess carbon dioxide to carbon monoxide, to adjust the temperature, to adjust the pressure, to separate the excess carbon dioxide, or a combination thereof.

17. The method of claim 1, wherein the syngas is conditioned by separating the excess carbon dioxide or diluent by a membrane, a pressure swing adsorber, a solvent-based separation system, or a combination thereof.

18. The method of claim 1, wherein the syngas is converted to methanol in a methanol synthesis unit.

19. The method of claim 18, wherein the methanol is subsequently converted to dimethyl ether (DME) in a one-step DME synthesis unit or a two-step DME synthesis unit.

20. (canceled)

21. The method of claim 1, wherein the syngas is converted to a lower olefin, a liquid fuel, or an aromatic in a hydrocarbon synthesis unit or a synthetic crude oil in a Fischer Tropsch (FT) reactor.

22. (canceled)

23. A system for the conversion of biogas to methanol, DME and/or hydrocarbons which comprises
(a) a biogas processing unit removing a substantial portion of sulfur compounds from the biogas and, optionally removing at least a portion of carbon dioxide from the biogas, to generate a clean biogas stream with about 1 to about 35 mol. % carbon dioxide content;
(b) an air separation unit to generate an oxygen-rich gas stream with about 25 to about 95 mol. % oxygen;
(c) an internal combustion engine reactor fluidly connected to the biogas processing unit and the air separation unit so as to react the clean biogas stream with the oxygen enriched stream so as to produce a syngas stream;
(d) a gas separation unit and a syngas compression unit fluidly connected to the syngas stream from the internal combustion engine reactor so as to generate a processed syngas stream; and
(e) a methanol, a DME, and/or a hydrocarbon synthesis unit fluidly connected to the processed syngas stream.

24. (canceled)

25. The system of claim 23, wherein the gas separation unit is fluidly connected to the internal combustion engine reactor to introduce carbon dioxide as a diluent in the internal combustion engine reactor.

26. The system of claim 23, where the methanol synthesis unit produces a hydrogen stream and the hydrogen stream is fluidly connected to the internal combustion engine reactor, the gas separation unit, the syngas compression unit, or a combination thereof.

27. A system for the conversion of natural gas to synthetic crude oil which comprises
(a) a natural gas fluid stream;
(b) an air separation unit to generate an oxygen-rich gas stream with about 25 to about 95 mol. % oxygen;
(c) an internal combustion engine reactor fluidly connected to the natural gas source and the air separation unit so as to react the clean biogas stream with the oxygen enriched stream so as to produce a syngas stream;
(d) a water gas shift/gas separation unit and a syngas compression unit fluidly connected to the syngas stream from the internal combustion engine reactor so as to generate a processed syngas stream and a carbon dioxide rich stream;
(e) wherein the carbon dioxide rich stream is fluidly connected to the internal combustion engine reactor to provide a carbon dioxide containing diluent stream; and
(f) a Fischer Tropsch (FT) reactor fluidly connected to the processed syngas stream.

28. The system of claim 27, wherein the water gas shift/gas separation unit is fluidly connected to the internal combustion engine reactor to introduce carbon dioxide as a diluent for the internal combustion engine reactor.

29. The system of claim 27, where the Fischer Tropsch (FT) reactor, the water gas shift/gas separation unit, the syngas compression unit, or a combination thereof, produces a hydrogen stream and the hydrogen stream is fluidly connected to a feed gas stream for the internal combustion engine reactor.

30. The system of claim 27, wherein the synthetic crude from the Fischer Tropsch (FT) reactor is fluidly connected to a crude upgrading unit and the crude upgrading unit produces diesel and/or naphtha.
